(19) United States
(12) Patent Application Publication
(10) Pub. No.: US 2022/0348853 A1
(43) Pub. Date: Nov. 3, 2022

(54) METHODS AND COMPOSITIONS FOR DELIVERING COMPACT LYOPHILIZED AGENTS FOR DISSOLVING IN A CLOSED SYSTEM

(71) Applicant: Bio-Techne Corporation, Minneapolis, MN (US)

(72) Inventors: Timothy John Manning, JR., Minneapolis, MN (US); Joy Lynn Aho, Maple Grove, MN (US); Todd Geders, Minneapolis, MN (US); Sean Henry Kevlahan, North Andover, MA (US); Garrett Michael Morris, St. Francis, MN (US); Charles D. Brawley, Minneapolis, MN (US)

(21) Appl. No.: 17/641,482

(22) PCT Filed: Sep. 9, 2020
(86) PCT No.: PCT/US2020/049908

§ 371 Date: Mar. 9, 2022

Related U.S. Application Data
(60) Provisional application No. 62/897,799, filed on Sep. 9, 2019.

(51) Int. Cl. C12M 1/00; B01L 3/00

(52) U.S. Cl. CPC C12M 23/14; C12M 23/26; B01L 3/52; B01L 2200/16; B01L 2300/123

(57) ABSTRACT

The invention relates to packaged lyophilized agents. In particular, the present invention relates to compact lyophilized agents that are packaged in flexible containers, and systems for their manufacture and use.

The present application claims priority to U.S. Provisional Application Ser. No. 62/897,799, filed Sep. 9, 2019, which is incorporated herein by reference.

### FIELD OF THE INVENTION

The invention relates to packaged lyophilized agents. In particular, the present invention relates to compact lyophilized agents that are packaged in flexible containers.

### BACKGROUND OF THE INVENTION

Current commercially available lyophilized agents, including proteins and small molecules, are provided in rigid containers, such as glass or plastic vials, which are unable to sterilely dock to closed systems. Accordingly, transfer of the lyophilized agent to the desired system requires manual reconstitution and transfer of the agent, thus increasing the chance of error, protein loss, contamination, and total labor time. These current processes are even more cumbersome when multiple agents need to be mixed and added to the closed system at the same time, thus requiring multiple times when the user must reconstitute the factor, aliquot the material into single-use vials, and then use the end solution.

Accordingly, what is needed are improved packaged lyophilized agents that can be efficiently and rapidly dissolved and transferred within closed systems without loss and/or contamination.

### SUMMARY OF THE INVENTION

Provided herein are systems and methods related to packaging and use of compact lyophilized agents, in particular, compact lyophilized agents housed within flexible containers. For example, in some embodiments, a system comprises at least one compact lyophilized agent housed within a flexible container. In some embodiments, the system comprises one compact lyophilized agent, while in other embodiments, the system comprises two or more compact lyophilized agents. In preferred embodiments, the flexible container is sterile.

The technology is not limited to any particular principal agent or agents (e.g., drugs, proteins, and the like) in the compact lyophilized agent. In certain embodiments, the at least one compact lyophilized agent comprises a protein or a small molecule. In certain preferred embodiments, the at least one agent comprises a protein selected from Flt-3 ligand, TPO, G-CSF, GM-CSF, IFN-gamma, IL-1 beta, IL-2, IL-3, IL-4, IL-6, IL-7, IL-10, IL-12, IL-15, IL-18, IL-21, M-CSF, Activin A, BMP-4, EGF, FGF basic, FGF-3, FGF-10, IGF-1, KGF/FGF-7, LIF, Noggin, PDGF-BB, R-Spondin 1 SCF/c-kit, Sonic Hedgehog, TGF-beta 1, VEGF 165, and Mouse LIF.

In some embodiments, one or more compact lyophilized agents further comprises a colorant. For example, in embodiments comprising two or more compact lyophilized agents, the two or more compact lyophilized agents may comprise the same colorants or they may comprise distinct colorants. In some embodiments, compact lyophilized agents comprising distinct colorants comprise different amounts of at least one agent and in some embodiments, compact lyophilized agents comprising distinct colorants comprise at least one different agent. In the latter embodiments, compact lyophilized agents comprising different agents are not limited by which agent is different. For example, in some embodiments, compact lyophilized agents comprising distinct colorants may comprise different principal agents, such as different proteins, yet may include the same excipients or other components, while in other embodiments, compact lyophilized agents comprising distinct colorants may contain the same principal agents and may differ in constituent components, e.g., they may comprise different excipients. In yet other embodiments, compact lyophilized agents comprising distinct colorants may contain the same principal component and same other constituent components, by may contain different amounts of any one of these components.

In some embodiments, the flexible container comprises a means, e.g., a port, tube, septum, for addition of a reconstitution fluid to the flexible container, preferably for aseptic addition of the fluid. In preferred embodiments, at least one compact lyophilized agent dissolves within one minute of the reconstitution fluid being added to the system.

In some embodiments, the system further comprises a means for delivering the reconstituted lyophilized agent from the flexible container to a second system, e.g., via a fluid connection. The second system is not limited to any particular form of second system. In certain preferred embodiments, the second system is a cell culture system, a bioreactor, a fermenter, or a subject, e.g., a mammal, preferably a human.

In some embodiments, the technology provides an aseptic closed system comprising a flexible container that houses at least one compact lyophilized agent; and a cell culture system containing a culture medium, wherein the flexible container is connected to the cell culture system at a fluid connection. In some embodiments, the cell culture system comprises a bioreactor or fermenter.

Fluid connection and fluid connectors are well known in the art, and the technology is not limited to any particular fluid connection. For example, in some embodiments, the fluid connection comprises tubing, and/or a connector such as a Luer connector. In certain embodiments, the fluid connection comprises weldable tubing.

The technology provides methods for making and using the systems described herein. In some embodiments, the technology provides a method comprising delivering at least one compact lyophilized agent into flexible container, then sealing the flexible container so as to contain the compact lyophilized agent. Preferably, the flexible container comprises a weldable bag comprising at least one port, and the sealing comprises welding the weldable bag. In certain embodiments, the weldable bag is gas-impermeable, while in some embodiments, the weldable bag is at least partially gas-permeable. In some embodiments, the flexible container is treated to reduce static charge on a surface of the flexible container. In preferred embodiments, the flexible container is treated to reduce static charge prior to delivery of the at least one compact lyophilized agent into flexible container.

In some embodiments, the technology provides a method, comprising delivering reconstitution fluid into a flexible container that houses at least one compact lyophilized agent to form reconstituted lyophilized agent; and delivering reconstituted lyophilized agent from the flexible container to a second system, e.g., a cell culture system, a bioreactor, a fermenter, or a subject. In some embodiments, the second system is a cell culture system that comprises a bag comprising a culture medium. In preferred embodiments, the flexible container and the second system are connected at a fluid connection. In some embodiments, the fluid connection comprises a Luer connector. In some embodiments, the fluid connection comprises tubing, and in certain preferred embodiments, the tubing comprises a welded connection.

The technology further provides a method comprising aseptically delivering reconstitution medium into a flexible container that houses at least one compact lyophilized agent to form reconstituted lyophilized agent and aseptically delivering a cell to the reconstituted medium in the flexible container. In certain embodiments, the method further comprises incubating the cell in the reconstituted medium, e.g., under conditions wherein a cell culture is grown from the cell. In preferred embodiments, the flexible container is at lease partially gas-permeable.

### Definitions

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the technology may be readily combined, without departing from the scope or spirit of the technology.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

The transitional phrase “consisting essentially of” as used in claims in the present application limits the scope of a claim to the specified materials or steps “and those that do not materially affect the basic and novel characteristic(s)” of the claimed invention, as discussed in In re Herz, 537 F.2d 549, 551-52, 190 USPQ 461, 463 (CCPA 1976). For example, a composition “consisting essentially of” recited elements may contain an unrecited contaminant at a level such that, though present, the contaminant does not alter the function of the recited composition as compared to a pure composition, i.e., a composition “consisting of” the recited components.

As used herein, the term “agent” refers to any suitable agent that may be lyophilized. For example, the agent may be a protein or a small molecule.

As used herein, the term “principal agent” refers to an agent (e.g., protein, small molecule) to be delivered as a compact lyophilized for an intended purpose or application (e.g., supplementation of a culture medium). A principal agent is contrasted, for example, with other agents, e.g., excipients, carriers, colorants, and the like, that may be included to give desired physical properties to a compact lyophilized agent (e.g., size, weight, shape, porosity, hardness, color) but that may be neutral or inert in the intended purpose of application for which the principal agent is to be used.

As used herein, the term “compact,” as used in reference to a lyophilized agent, refers to a lyophilized agent that has been produced to be bound within a compact form, e.g., a shape. For example, a “compact lyophilized agent” may refer to a lyophilized agent that has been produced to have particular, e.g., a spherical shape. In some embodiments, producing an agent to have a shape comprises arranging the agent in a wet form (e.g., dissolved, suspended, moistened) into a shape, e.g., a drop or mound, prior to lyophilization, such that a desired form is maintained during, or is achieved by, a process of lyophilization. In other embodiments, reagents for producing a compact lyophilized agent are shaped during and/or after a lyophilization process, e.g., by compressing, molding, 3D printing, etc.

A “compact” lyophilized agent is also referred to interchangeably herein as a “dot.” A “dot” is not limited to any particular shape of compact lyophilized reagent and may be, for example, a sphere, spheroid, cube, polyhedron, etc., or an irregular form, e.g., like a bean, pea, etc. In preferred embodiments, a dot is generally spherical.

An individual unit of compact lyophilized agent, e.g., an individual sphere or dot, is typically configured to comprise a defined amount of the agent, e.g., protein or small molecule, such that a defined amount of an agent may be selected (e.g., for adding to a volume of fluid) by selecting the correct number of individual spheres for addition to the volume of fluid. By way of example and not by way of limitation on the scope of the technology, ProDots® spheres may be manufactured to contain 5 μg of protein in each sphere, such that adding 10 μg of the protein to a fluid required adding two spheres to the fluid. As discussed further below, it is understood that a compact lyophilized agent may be manufactured to contain any amount of agent. The term “ProDots®” as used herein may refer to a single unit of a compact lyophilized agent, e.g., a single dot or sphere, or to a plurality of units, e.g., a plurality of dots or spheres.

As used herein, the term “distinct” as in reference to features of compact lyophilized agents (e.g., the shape, composition, colorant, etc.) refers to features that can be differentiated one from another between two or more different compact lyophilized agents. Such “distinct” features may allow differentiation e.g., by visual inspection (e.g., of color, shape, size) or by analysis of data related to characteristics of the compact lyophilized agents (e.g., by formula data used in preparation of the compact lyophilized agent, or by analytical results from tests conducted on the compact lyophilized agent.)

Advantageously, compact lyophilized agents may be manufactured in bulk, and the individual units may be subsequently packaged to provide a known quantity of the agent in a package according to the number of individual units of the compact lyophilized agent, without further weighing, measuring, aliquoting, etc.

As used herein, the term “closed system” refers to a system designed such that the product is not exposed to the room environment. For example, a closed system may refer to a closed cell culture system.

As used herein, the term “functionally closed system” refers to a system that may be opened but is returned to a functionally closed state through a sanitization, sterilization, or filtration step after opening. For example, a cell culture system may be a functionally closed cell culture system.

As used herein, the terms “flexible container” or “flexible storage container” as used herein refer to any suitable flexible container in which the lyophilized agent may be housed. The term “flexible” when used in reference to a container refers to a container that is capable of having the shape of the space contained within the container readily deformed (e.g., by bending, twisting, folding, etc.), under modest force, e.g., by a hand or hands. The flexibility of the flexible container is contrasted with deformability of rigid containers that cannot be readily deformed by hand (e.g., of metal, or of plastic that is thick enough to be rigid, etc.), even though such rigid containers may nonetheless be deformed by application of a large force, e.g., by tool such as a hammer or pliers. The term “flexible container” is used throughout in order to differentiate between rigid containers (e.g., glass or hard plastic vials, tubes, assay plates, bottles, etc.), which are known in the art and which have several disadvantages over the disclosed systems.

As used herein, the term “reconstitution fluid” refers to any suitable liquid that can be used to dissolve a lyophilized agent, including but not limited to buffers, acidic, pH neutral, or basic solutions, culture media, serum (e.g., human serum, fetal bovine serum), etc. Accordingly, a “reconstitution fluid” may be a reconstitution buffer, reconstitution solution, reconstitution medium, reconstitution serum, etc.

As used herein, the term “weld” as used in reference to components, e.g., plastic components, refers to joining or fusing of surfaces of the components. Welding generally comprises softening surfaces to be joined, contact of the softened surfaces together under pressure, and hardening of the softened junction such that the surfaces are bonded or fused. Typically, welding of plastics is performed by the application of heat and pressure, but plastics may also be welded chemically, e.g., using a solvent, using cross-linking, e.g., photo-crosslinking, etc.

The term “weldable” as used herein refers to an ability of a component to be joinable in a welded fashion, by forming a weld between components or parts of the components. For example, a “weldable bag” may refer to a bag that may be closed (e.g., sealed) by formation of a welded seam, e.g., between sides or portions of the bag. A weldable bag may also refer to a bag that is joinable to another component, e.g., a weldable tube or a different weldable bag, by forming a weld between the bag and the tube.

As used herein, the term “joinable” refers to the ability of a component of a system disclosed herein to connect to a different component of a system. For example, the term “joinable” may be used to refer to a component of a first system that is able to connect to a different component of the first system. Alternatively, the term “joinable” may be used to refer to a component of a first system that is able to connect to a component of a different system. In some embodiments, components are joinable in a weldable fashion, by forming a weld between components or parts of the components.

### DESCRIPTION OF THE DRAWINGS

These and other features, aspects, and advantages of the present technology may become better understood by reference to the following drawings:

FIG. 1A is a picture showing an embodiment of the flexible container described herein.

FIG. 1B is a schematic diagram of an embodiment of a flexible container described herein.

FIG. 2 is a schematic diagram showing various embodiments of the packaged compact lyophilized agents described herein. The compact lyophilized agent may be stored in a sterile, flexible container (e.g., a bag). A container may hold one type of compact lyophilized agent (left panel, showing circles representing three different proteins (P #1, P #2, and P #3) in separate flexible containers). Alternatively, a flexible container may hold multiple, distinct compact lyophilized agents (right panel).

FIG. 3A-3B show RP-HPLC results after dissolving a compact lyophilized dot of recombinant human IL-7 (rhIL-7) dot in reconstitution buffer. Reverse phase 280 nm absorbance traces of rhIL-7 at times t=0 and t=30 minutes post reconstitution are shown. 50 μL of sample at 0.1 mg/mL were loaded to Agilent C3 Reverse Phase and separated using a water/TFA to acetonitrile/TFA gradient. Samples tested are rhIL-7 ProDots® compact lyophilized IL-7 deriving from Bulk Lot GAAS02 immediately upon reconstitution (FIG. 3A) and 30 minutes afterwards (FIG. 3B).

FIG. 4A-4B show mass spectrometry results after dissolving a compact lyophilized dot of recombinant human IL-7 dot in reconstitution buffer. 50 μL of sample at 0.1 mg/mL were loaded to Agilent C3 Reverse Phase and separated using a water/TFA to acetonitrile/TFA gradient. Samples tested are bagged rhIL-7 ProDots® deriving from Bulk Lot GAAS02 immediately upon reconstitution (FIG. 4A) and 30 minutes afterwards (FIG. 4B).

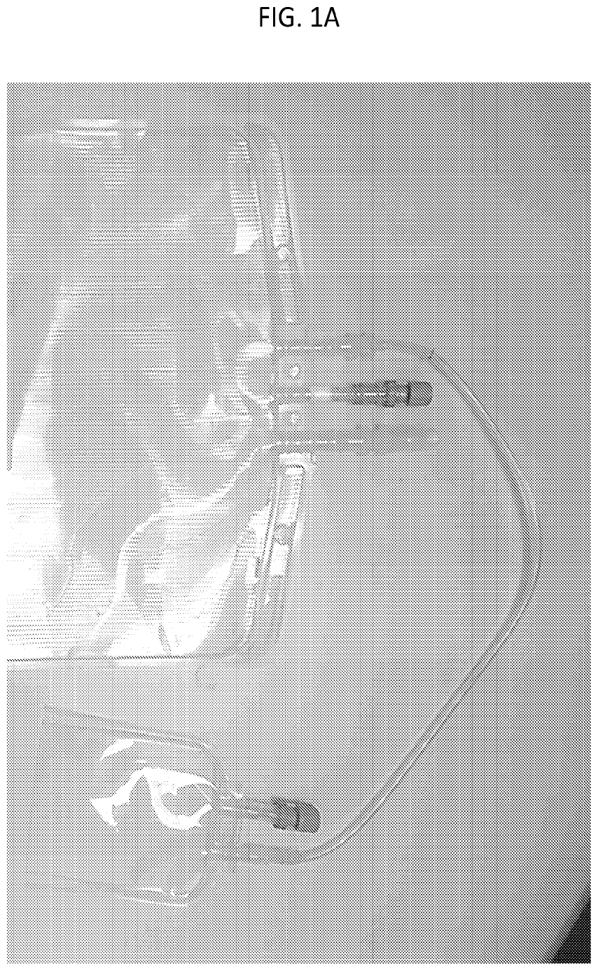

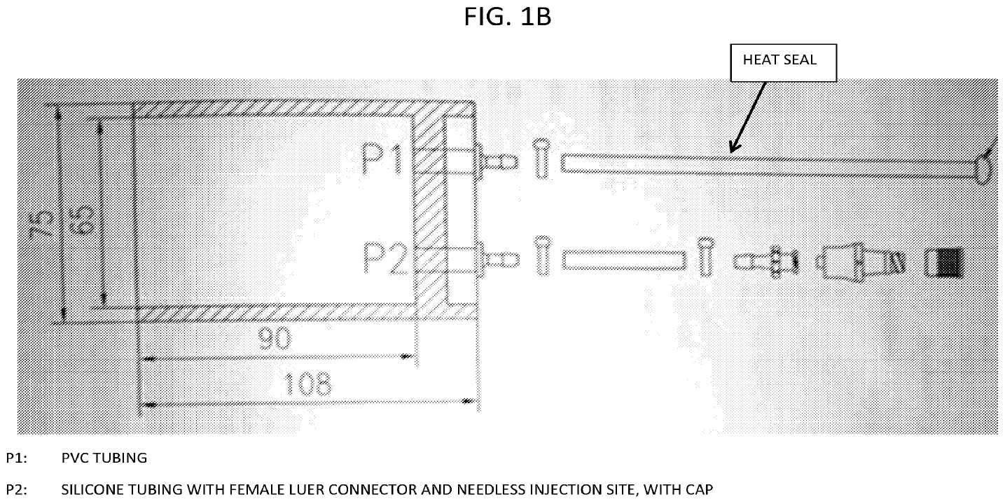

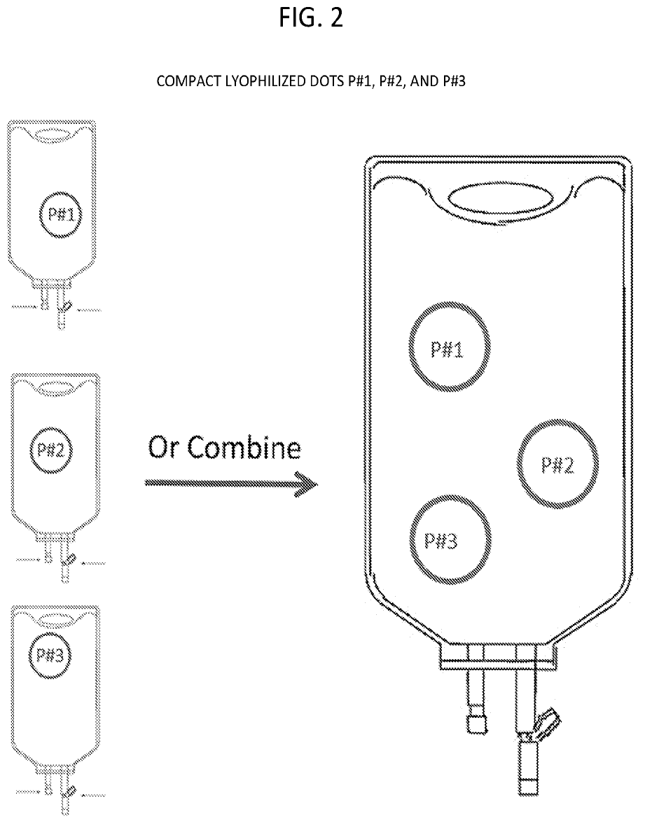

### DETAILED DESCRIPTION OF THE INVENTION

The technology relates to packaged lyophilized agents. In particular, the technology relates to compact lyophilized agents that are packaged in flexible containers.

The systems described herein are advantageous in that the systems provide a compact lyophilized agent in a ready-to-use flexible storage container that allows for rapid reconstitution of the agent and direct transfer to the second system without the need for manual reconstitution and aliquoting steps. Maintaining sterility is critical to many systems, including cell culture systems and therapeutic systems and accordingly any open, manipulation increases the risk of contamination. The systems provided herein eliminate open manipulation and lower the chance for contamination.

In some embodiments, provided herein are systems comprising at least one compact lyophilized agent housed within a flexible container. In preferred embodiments, essentially all lyophilized agent housed within the flexible container is within one or more units of the at least one compact lyophilized agent. In some embodiments, the system comprises one compact lyophilized agent. In other embodiments, the system comprises two or more compact lyophilized agents. For example, the system may comprise two compact lyophilized agents, three compact lyophilized agents, four compact lyophilized agents, five compact lyophilized agents, six compact lyophilized agents, seven compact lyophilized agents, eight compact lyophilized agents, nine compact lyophilized agents, or ten or more compact lyophilized agents. The number and selection of the compact lyophilized agents will depend on the intended use of the system (e.g., for cell culture methods, therapeutic methods, etc.).

The at least one compact lyophilized agent may be any suitable agent. For example, the agent may comprise a suitable protein or small molecule. In some embodiments, the agent comprises a protein. In other embodiments, the agent comprises a small molecule. The specific agent will depend on the intended use of the system. For example, for systems intended for use in cell culture methods, any suitable protein and/or small molecule for cell culture may be selected. Exemplary proteins for use in cell culture include, but are not limited to, Flt-3 Ligand, G-CSF, GM-CSF, TPO, IFN-gamma, IL-1 beta, IL-2, IL-3, IL-4, IL-6, IL-7 IL-10, IL-12, IL-15, IL-18, IL-21, M-CSF, Activin A, BMP-4, EGF, FGF basic, FGF-3, FGF-10, IGF-1, KGF/FGF-7, LIF, Noggin, PDGF-BB, R-Spondin 1 SCF/c-kit, Sonic Hedgehog, TGF-beta 1, VEGF 165, and Mouse LIF. As another example, for systems intended for use in therapeutics, the agent may comprise any suitable protein and/or small molecule for treatment of a disease or condition in a subject. In some embodiments, the at least one compact lyophilized agent is a ProDots® protein.

The compact lyophilized agent may comprise any suitable amount of the agent. Suitable amounts will depend on, for example, the intended use of the system. In some embodiments, the compact lyophilized agent may comprise 1 ng to 100 g of the agent. For example, the compact lyophilized agent may comprise 1 ng, 5 ng, 10 ng, 20 ng, 30 ng, 40 ng, 50 ng, 60 ng, 70 ng, 80 ng, 90 to 999 ng; or any amount therebetween; 1 μg, 5 μg, 10 μg, 20 μg, 30 μg, 40 μg, 50 μg, 60 μg, 70 μg, 80 μg, 90 to 999 μg, or any amount therebetween; 1 mg, 5 mg, 10 mg, 20 mg, 30 mg, 40 mg, 50 mg, 60 mg, 70 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 900 mg, 1 g, 10 g, 20 g, 30 g, 40 g, 50 g, 60 g, 70 g, 80 g, 90 g, or 100 g of the agent. The above examples are not intended to be limiting, any suitable defined amount of the lyophilized agent may be used.

In some embodiments, the at least one compact lyophilized agent further comprises one or more carriers or excipients. The terms “carrier” and “excipient” are used interchangeably herein, and refer to agents that facilitate any aspect of working with compact lyophilized agents, e.g., production, storage, handling, packaging, transport, stabilizing, solubility, bioavailability, etc. In some embodiments, a carrier is used to provide additional bulk or mass, such that a desired small amount of a principal agent, e.g., a protein, can be formed into a compact lyophilized form having particular desired properties, e.g., size, weight, shape, porosity, hardness, color, for a particular intended use. Such carriers may be added to the principal agent prior to lyophilization and formation of the compact form. Suitable carriers include, for example, BSA, HSA, sucrose, lactose, trehalose, mannitol, surfactants, phospholipids, and the like. In some embodiments, the compact lyophilized agent does not comprise any carriers (e.g., the compact lyophilized agent consists of pure lyophilized protein).

In some embodiments, the at least one compact lyophilized agent further comprises an indicator, e.g., a colorant. See, e.g., Allam, et al., International Journal of Pharmacy and Pharmaceutical Sciences Vol 3, Suppl 3, 201, which is incorporated herein by reference in its entirety. In preferred embodiments, the indicator is colored pigment or dye. A colorant may be natural (e.g., a plant-derived colorant, such as an anthocyanidin) or may be synthetic. A colorant may, for example, be used to indicate the type and/or quantity of agent housed within the system. In some embodiments, a colorant may be used to indicate which specific protein and/or small molecule is present. Any suitable color of colorant may be used, including red, orange, yellow, green, blue, purple, black, gray, etc. In some embodiments, a colorant may be detectable by detecting fluorescence. In some embodiments, the color of the colorant may be selected to allow for easy visual confirmation that the appropriate number and/or type of compact lyophilized agent(s) are present in the flexible container.

In some embodiments, the system comprises two or more compact lyophilized agents and each agent comprises a colorant. Any suitable colorant or combination of colorants may be used. For example, the system may comprise two or more compact lyophilized agents that comprise the same protein and/or small molecule. In this instance, each of the two or more agents may further comprise the same colorant (e.g. blue, red, green, yellow, purple, black, etc.). Alternatively, the system may comprise two or more compact lyophilized agents, wherein at least one of the compact lyophilized agents is distinct from another agent (e.g., one agent comprises protein X, the other agent comprises protein Y). In such embodiments, the agent comprising protein X may comprise a first colorant and the agent comprising protein Y may comprise a second colorant. Accordingly, the user is able to visually confirm that the correct number and types of compact lyophilized agents are present in the system. Similarly, in some embodiments, one compact lyophilized agent comprises a first amount of protein X and the other compact lyophilized agent comprises a different amount of protein X, and the agent comprising the first amount of protein X may comprise a first colorant and the agent comprising the second amount of protein X may comprise a second colorant. Accordingly, the user is able to visually confirm that a correct total amount of protein X present in the system by confirming the correct numbers of each color of compact lyophilized agents are present in the system.

The at least one compact lyophilized agent may be any suitable size and shape. In some embodiments, the compact lyophilized agent may be a spherical shape. In some embodiments, the compact lyophilized agent may be a size that can visualized without the use of a microscope. For example, the compact lyophilized agent may be at least 1 mm in diameter. For example, the compact lyophilized agent may be a spherical shape of about 1 mm to about 10 cm in diameter. For example, the compact lyophilized agent may be a spherical shape of about 1 mm, 2 mm, 3 mm, 4 mm, 5 mm, 6 mm, 7 mm, 8 mm, 9 mm, 10 mm, 2 cm, 3 cm, 4 cm, 5 cm, 6 cm, 7 cm, 8 cm, 9 cm, or 10 cm in diameter. In some embodiments, the compact lyophilized agent may be larger than 10 cm in diameter.

In accordance with any of the embodiments described herein, the at least one compact lyophilized agent is housed within a flexible container. The compact lyophilized agent may be housed within the flexible container without being adhered to an interior surface of the flexible container. For example, the compact lyophilized agent may be moveable within the flexible container. Allowing the compact lyophilized agent to be movable within the flexible container may enable shipping or transporting of the system with minimal risk of damage to the compact lyophilized agent, such as minimal risk of breaking the compact lyophilized reagent.

The flexible container may be any suitable size or shape. In some embodiments, the flexible container is a bag. In some embodiments, the flexible container is a weldable bag. In some embodiments, the flexible container may be sterile. Sterility may be achieved by any suitable method as known in the art, including dry heat, autoclaving (e.g. steam sterilization), ethylene oxide, irradiation, gamma-ray, e-beam, supercritical CO2 and the like.

The flexible containers described herein may comprise any suitable material or combination of suitable materials. Suitable materials include, for example, polyethylene, ethylene vinyl alcohol, silicone, polycarbonate, FEP (Fluoroethyl polymer); EVA (Ethylene vinyl acetate), ULDPE (ultra low-density polyethylene), and the like. The flexible container may comprise one layer or multiple layers. For example, the flexible container may be a single layer bag or a multi-layer bag. In particular embodiments, the flexible container may comprise multilayer films including one or more of polyethylene, polyester, ethylene vinyl alcohol EVOH, and ultra low-density polyethylene.

In some embodiments, the flexible container may be gas permeable. In other embodiments, the flexible container may be gas impermeable. In some embodiments, a flexible container may comprise gas-permeable portions and gas impermeable portions. For example, in some embodiments, a compact lyophilized agent may be packaged within a gas-impermeable area of or within a flexible container, such that the agent can be manipulated into a gas-permeable portion of the container. In some embodiments, the agent is dissolved or suspended before manipulation into a gas-permeable portion of a flexible container, and in some embodiments, the agent is manipulated into a gas-permeable portion of the flexible container while still in a lyophilized form.

In some embodiments, the flexible container may be light blocking. For example, in embodiments, wherein the agent is photo labile, the flexible container may be partially or completely light blocking to prevent degradation of the agent.

In some embodiments, the flexible container may be sealed within a suitable packaging. For example, the system may be sealed within a vacuum-sealed pouch, such as an aluminum pouch. In some embodiments, the packaging may include a desiccant. In some embodiments, the packaging may be light blocking. In particular embodiments, the flexible container may be sealed within a vacuum-sealed foil pouch. In such embodiments, the user may open the packaging prior to use of the system. In particular embodiments, the user may open the packaging and clean and/or sterilize the exterior of the flexible container prior to use. For example, the user may open the packaging and sterilize the exterior of the flexible container with a suitable solution (e.g. isopropanol solution) prior to use.

In some embodiments, the flexible container further comprises a means for addition of a reconstitution fluid to the flexible container to allow for dissolution of the at least one compact lyophilized agent housed within the flexible container. For example, the flexible container may further comprise a port for tubing (e.g. weldable tubing). In some embodiments, the port allows for two-way flow of liquid (e.g., flow of liquid into and out of the flexible container). In some embodiments, tubing can be connected to the port of the flexible container by a suitable method to allow for addition of a reconstitution buffer to the tubing and subsequent flow from the tubing into the flexible container and/or flow from the flexible container back into the tubing. As another example, the flexible container may further comprise a port that allows for the direct addition of reconstitution fluid to the flexible container through a syringe, pipette, needle, and the like. In accordance with any of these embodiments the port may be sealed when not in use by any suitable means, including caps, stoppers, lids, etc. Alternatively, the flexible container may comprise a port that is able to be punctured (e.g. by a needle) to allow for direct addition of the reconstitution fluid to the flexible container through a syringe.

In some embodiments, the flexible container further comprises a means for delivering the reconstituted lyophilized agent from the flexible container to a second system. In some embodiments, the second system may be a closed system. The closed system may be any system in which the reconstituted agent is not exposed to the ambient environment. For example, the closed system may be a closed cell culture system. In some embodiments, the second system may be a functionally closed system, such as a functionally closed cell culture system. In some embodiments, the flexible container may further comprise a means for delivering the reconstituted lyophilized agent from the flexible container to the closed or functionally closed cell culture system. For example, the flexible container may further comprise a port for weldable tubing that may be connected to a component of the cell culture system to allow for transfer of the reconstituted lyophilized agent from the flexible container to the component of the cell culture system. Suitable components of the closed cell culture system include, for example, flasks, vials, tubes, plates, dishes, bags, cell culture media, tubing, and the like. In some embodiments, the second system may be a bioreactor. In some embodiments, the second system may be a fermenter.

In some embodiments, the second system comprises a suitable container holding cell culture media. For example, the second system may be a culture media bag. In such embodiments, the flexible container may comprise a port for weldable tubing. Accordingly, the flexible container may be connected to the culture media bag by the weldable tubing. The user may manually manipulate the culture medium through this connection to the flexible container to solubilize the compact lyophilized agent. The user may subsequently manually manipulate the reconstituted agent back to the culture medium bag through this connection. The fluid manipulation between the flexible container and the second system may be repeated multiple times as necessary. For example, the fluid manipulation may be repeated 4-5 times to ensure that all of the dissolved agent (e.g., protein) is moved to the second container (e.g., the culture media bag). The fluid in the culture media bag may then be transferred via sterile tubing to the desired component of the cell culture system. The fluid manipulation may be performed manually, e.g., by a subject or worker, or by use of an automated system. For example, an automated system may comprise a peristaltic, positive, or negative pressure pump flowing liquid in either a laminar or turbulent manner.

In some embodiments, the second system may be a subject. In some embodiments, the subject may be a mammal. For example, the subject may be a human. The flexible container may further comprise a means for delivering the reconstituted lyophilized agent from the flexible container to the subject. For example, the flexible container may further comprise a port for tubing (e.g., weldable tubing), that can be connected to the flexible container. The tubing may be connected to a needle or a syringe which can be placed into the subject (e.g., parenterally) to allow for transfer of the reconstituted lyophilized agent from the flexible container through the tubing and into the subject. For example, the needle may be placed into the subject intravenously, subcutaneously, intramuscularly, and the like. In some embodiments the tubing may be configured for fluid connection to a system for delivering fluids to a subject, e.g., an intravenous (I.V.) system. For example, in some embodiments, the tubing comprises a connector, e.g., a Luer connector, for connection to an I.V. system that is connected to the subject, such that reconstituted lyophilized agent is delivered to the subject via the I.V. system.

The systems described herein display several advantages over the art. In some aspects, the systems described herein are advantageous in that they allow for rapid dissolution of the compact lyophilized agent housed within the flexible container and are immediately ready for use. In some embodiments, the at least one compact lyophilized agent dissolves within one minute of the reconstitution buffer being added to the system. For example, the at least one compact lyophilized agent may dissolve in less than 60 seconds, less than 55 seconds, less than 50 seconds, less than 45 seconds, less than 40 seconds, less than 35 seconds, less than 30 seconds, less than 25 seconds, less than 20 seconds, less than 15 seconds, less than 10 seconds, less than 5 seconds, less than 2.5 seconds, or less than 1 second after addition of the reconstitution buffer to the flexible container.

In other aspects, the systems described herein are advantageous in that the compact lyophilized agent(s) display minimal adherence to the flexible container. This is in contrast to traditional lyophilized agents, e.g., a lyophilized cake formed in a vial, which may be completely or partially adhered to the vial in which it is packaged. Accordingly, the systems described herein allow for dissolution of the compact lyophilized agent and transfer of the reconstituted agent to the desired system without loss of the agent (e.g., without loss of protein or small molecule). In preferred embodiments, compact lyophilized agents of the technology can be moved from container to container, or moved within a container by use of external forces, e.g., gravity. In preferred embodiments, compact lyophilized agents of the technology are droppable (e.g., can be placed in packaging by dropping into the package) and rollable (e.g., will roll on a sloped surface, for example into or out of a flexible package, or between different points within a flexible package, by force of gravity).

### EXPERIMENTAL EXAMPLES

### Example 1

Input Materials:
Lyophilized ProDots® containing rhIL-7 GMP Bulk Lot GAAS02 (5 μg per ProDot)
Weldable, sterile bags with appropriate fittings
Weldable foil pouch for final storage (tear-open with resealable Zipper Tamper Evident (ZTE) ZipSeal™ bags)

Material Preparation:

ProDots® of rhIL-7 GMP from Bulk Lot GAAS02 were formulated, lyophilized, and packaged into weldable bags for dissolution and functional testing.

Test Sample generated for analysis:
ProDots (rhIL-7) in Weldable Bags within Vacuum-sealed Foil Pouches
   1×5 μg ProDot per bag—OR—
   5×5 μg ProDot per bag

Experimental Design:

Materials were removed from the recommended storage condition, allowed to equilibrate to room temperature. Any external packaging (e.g., foil pouch or aluminum crimp seals) was removed and the product was reconstituted by adding sufficient volume of Dulbecco's Phosphate-Buffered Saline (PBS) to yield a final concentration of 100 μg/mL.

Analytical Testing by RP-HPLC and Mass Spectrometry:

ProDots® dissolve within seconds to <1 minute of reconstitution buffer being added. In contrast, traditional lyophilized products, e.g., in the form of a cake at the bottom of a glass vial (made from liquid lyophilized in the vial; see, e.g., 207-GMP-025 at www<dot>mdsystems<dot>com/products/good-manufacturing-practices-gmp-grade-proteins), are designed to fully dissolve within 30 minutes after reconstitution buffer is added.

Accordingly, analytical testing by reverse phase high performance liquid chromatography (RP-HPLC) and mass spectrometry was done at two time-points after reconstitution buffer was added: <1 minute (termed t=0 min) and 30 minutes (t=30 min). Due to mass requirements for testing, all containers tested (ProDots® in welded bags, and traditional lyophilized protein in vials) contained 25 μg total product.

TABLE 1
Peak recovery of 5 ug loads in C3 reverse phase of Recombinant
Human IL-7 after reconstitution to 0.1 mg/mL. Samples
were tested at t = 0 and t = 30 minutes.

| | | Peak | | | |
|---|---|---|---|---|---|
| | Sample | Retention | Peak Area | | |
| Sample | Type | (min) | (280 nm) | Time | |
| rhIL-7 GAAS02 | In Bag | 8.949 | 89.318 | 0 | min |
| ProDots ® | | | | | |
| rhIL-7 GAAS02 | In Bag | 8.947 | 88.248 | 30 | min |
| ProDots ® | | | | | |

### Summary of RP-HPLC Results:

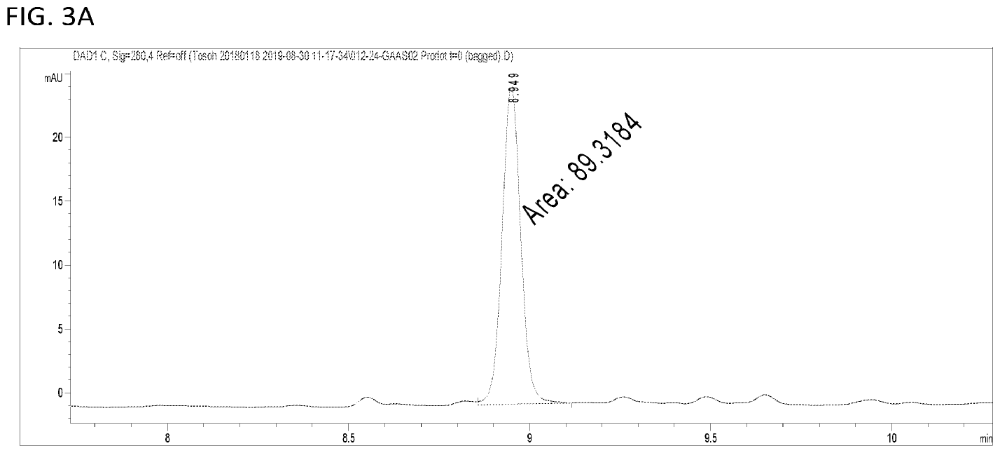

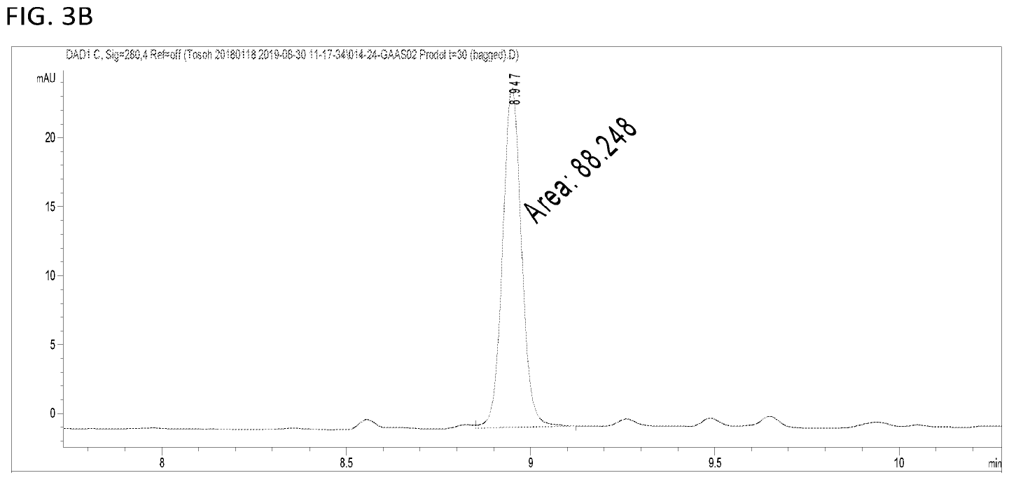

As shown in FIG. 3A-3B and Table 1, no difference in reconstitution between t=0 min and t=30 minutes was observed for the ProDots® sample. This suggests that reconstitution is very fast and that no further material is dissolved after 30 additional minutes of incubation with reconstitution buffer.

Summary of Mass Spectrometry results:

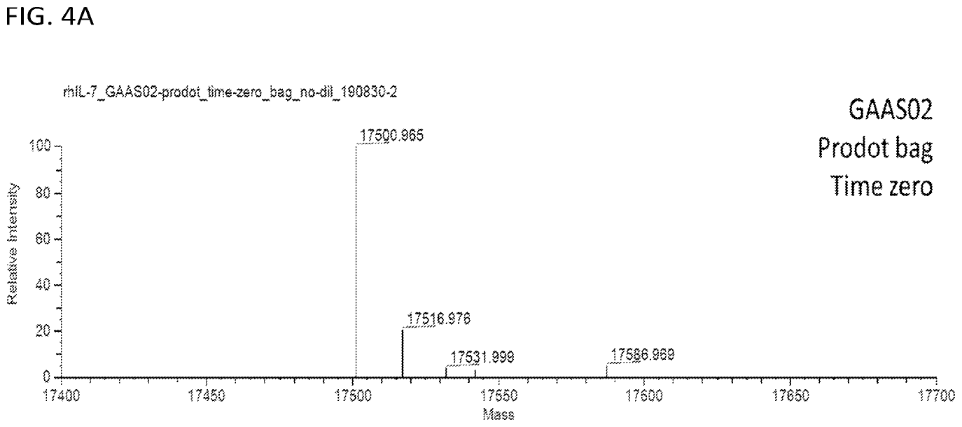

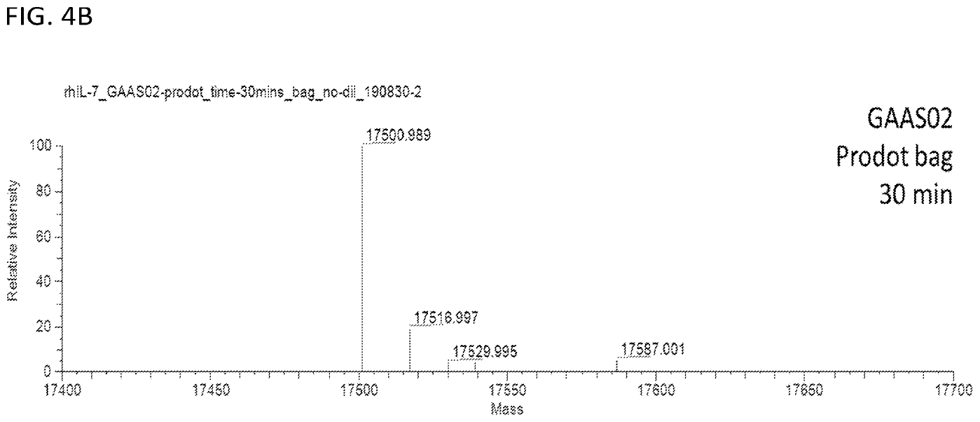

As shown in FIG. 4A-4B, all samples showed nearly indistinguishable spectra between ProDots® in bags at the two timepoints. The expected mass under these testing conditions is 17500.930 Da, and all materials tested matched this well for the major peak and was well within the accuracy of the Thermo Scientific™ Q Exactive™ HF instrument. Accordingly, the bioactive ingredient, rhIL-7, is unaltered based on any treatment or intermediate preparation. Moreover, this methodology allows for clear confirmation of the identity of the bioactive ingredient in a ProDots®. Accordingly, this methodology allows for distinguishing between different bioactive agents, such as between (e.g., rhIL-7 & rhIL-2).

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control.

Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in pharmacology, biochemistry, medical science, or related fields are intended to be within the scope of the following claims.

1. A system comprising at least one compact lyophilized agent housed within a flexible container.

2. The system of claim 1, wherein the system comprises one compact lyophilized agent.

3. The system of claim 1, wherein the system comprises two or more compact lyophilized agents.

4. The system of any one of claims 1-3, wherein the at least one compact lyophilized agent comprises a protein or a small molecule.

5. The system of claim 4, wherein the at least one agent comprises a protein selected from Flt-3 ligand, TPO, G-CSF, GM-CSF, IFN-gamma, IL-1 beta, IL-2, IL-3, IL-4, IL-6, IL-7, IL-10, IL-12, IL-15, IL-18, IL-21, M-CSF, Activin A, BMP-4, EGF, FGF basic, FGF-3, FGF-10, IGF-1, KGF/FGF-7, LIF, Noggin, PDGF-BB, R-Spondin 1 SCF/c-kit, Sonic Hedgehog, TGF-beta 1, VEGF 165, and Mouse LIF.

6. The system of any one of claims 1-5, wherein the at least one compact lyophilized agent further comprises a colorant.

7. The system of any one of claims 3-6, wherein each of two or more compact lyophilized agents comprises a colorant.

8. The system of claim 7, wherein two or more compact lyophilized agents comprise distinct colorants.

9. The system of claim 8, wherein compact lyophilized agents comprising distinct colorants comprise different amounts of at least one agent.

10. The system of claim 8, wherein compact lyophilized agents comprising distinct colorants comprise at least one different agent.

11. The system of any one of claims 1-10, wherein the flexible container is sterile.

12. The system of any one of claims 1-11, wherein the flexible container comprises a means for addition of a reconstitution fluid to the flexible container.

13. The system of claim 12, wherein the at least one compact lyophilized agent dissolves within one minute of the reconstitution fluid being added to the system.

14. The system of claim 12 or claim 13, wherein the system further comprises a means for delivering the reconstituted lyophilized agent from the flexible container to a second system.

15. The system of claim 14, wherein the second system is a cell culture system, a bioreactor, a fermenter, or a subject.

16. The system of claim 15, wherein the subject is a mammal.

17. The system of claim 16, wherein the subject is a human.

18. An aseptic closed system comprising:
i) a flexible container that houses at least one compact lyophilized agent; and
ii) a cell culture system containing a culture medium,
wherein the flexible container is connected to the cell culture system at a fluid connection.

19. The system of claim 18, wherein the cell culture system comprises a bioreactor or fermenter.

20. The system of claim 18 or claim 19, wherein the fluid connection comprises weldable tubing.

21. A method, comprising:
i) delivering at least one compact lyophilized agent into flexible container; and
ii) sealing the flexible container,
wherein the flexible container comprises a weldable bag comprising at least one port, and wherein the sealing comprises welding the weldable bag.

22. The method of claim 21, wherein the weldable bag is gas-impermeable.

23. A method, comprising:
i) delivering reconstitution fluid into a flexible container that houses at least one compact lyophilized agent to form reconstituted lyophilized agent; and
ii) delivering reconstituted lyophilized agent from the flexible container to a second system.

24. The method of claim 23, wherein the second system is a cell culture system, a bioreactor, a fermenter, or a subject.

25. The method of claim 23, wherein the cell culture system comprises a bag comprising a culture medium.

26. The method of claim 23, wherein the flexible container and the second system are connected at a fluid connection.

27. The method of claim 26, wherein the fluid connection comprises a Luer connector.

28. The method of claim 26 or claim 27, wherein the fluid connection comprises tubing.

29. The method of claim 28, wherein the tubing comprises a welded connection.

30. A method, comprising:
i) aseptically delivering reconstitution medium into a flexible container that houses at least one compact lyophilized agent to form reconstituted lyophilized agent; and
ii) aseptically delivering a cell to the reconstituted medium in the flexible container.

31. The method of claim 30, further comprising incubating the cell in the reconstituted medium.
